SEQUENCE LISTING (1 sequence)

SEQ ID NO: 1 - protein, 7 aa, Homo sapiens
DRVYIHP

(19) United States
(12) Patent Application Publication
(10) Pub. No.: US 2010/0055147 A1
(43) Pub. Date: Mar. 4, 2010

(54) ANGIOTENSIN (1-7) ELUTING STENT

(76) Inventors: Edze Jan Tijsma, Maastrich (NL); Anita Driessen-Levels, Reuver (NL)

Correspondence Address: K&L Gates LLP, 1900 MAIN STREET, SUITE 600, IRVINE, CA, 92614-7319, US

(21) Appl. No.: 12/447,415

(22) PCT Filed: Oct. 26, 2007
(86) PCT No.: PCT/US07/82708

§ 371 Date: May 18, 2009

Related U.S. Application Data
(60) Provisional application No. 60/863,270, filed on Oct. 27, 2006.

(51) Int. Cl. A61F 2/00; A61K 39/395; A61K 38/08; A61P 9/00

(52) U.S. Cl. 424/423; 424/130.1; 514/16

(57) ABSTRACT

Medical devices with polymer coatings designed to control the release of bioactive agents in combination with angiotensin-(1-7) receptor agonists from medical devices are disclosed. Methods for treating or inhibiting post-stent implantation restenosis as well as improving vascular endothelial function in patients are also provided.

### CROSS REFERENCE TO RELATED APPLICATIONS

This application claims the benefit under 35 U.S.C. §119(e) to U.S. Provisional Patent Application No. 60/863,270 filed Oct. 27, 2006 and is a continuation-in-part of U.S. patent application Ser. No. 11/256,852 filed Oct. 21, 2005, now U.S. Pat. No. 7,176,261, which claims the benefit under 35 U.S.C. §119(e) to U.S. Provisional Patent Application No. 60/621,462 filed Oct. 21, 2004; all of which are incorporated by reference herein in their entirety.

### FIELD OF THE INVENTION

This invention relates generally to vascular stents with controlled-release drug-eluting polymer coatings capable of inhibiting restenosis and improving vascular endothelial cell function. Specifically, the present invention provides for stents comprising bioactive agents preventing restenosis after vascular stent implantation by delivering these anti-restenotic compounds to the treatment site. More specifically, the present invention provides for improvement of the vascular endothelial cell function by delivering to the treatment site agonists of the angiotensin-(1-7) receptor. Furthermore, the present invention provides release of bioactive agents and angiotensin-(1-7) receptor agonists from coated stents to inhibit restenosis and improve vascular endothelial cell function.

### BACKGROUND OF THE INVENTION

In-stent restenosis (ISR), the renarrowing of blood vessels after angioplasty with stent placement, is an ongoing clinical problem, affecting an increasing number of patients as the amount of interventions increase. The advent of the drug-eluting stent (DES) was an important victory for the interventional cardiologist in the battle against ISR. Stents releasing the cytostatic compounds sirolimus or paclitaxel have lowered the incidence of clinical and angiographic restenosis and the need for reintervention by at least 50% as compared to bare metal stents. However, the incidence of death or myocardial infarction after stenting has not been improved, illustrating the progressive nature of the underlying vascular disease.

Further improvement might be achieved if stents release compounds that improve vascular and cardiac function. Preferably these compounds would inhibit neointima formation, to reduce restenosis through neointima formation, but also improve cardiac and endothelial function. The endothelium has been recognized as an important locus for cardiovascular intervention. Dysfunction of the endothelium, when defined as a decreased capacity to dilate arteries, has been associated with increased risk for atherosclerosis and worse prognosis in coronary artery disease (reviewed by Valgimigli et al. 2003). Recently, it was determined that the heptapeptide angiotensin-(1-7) not only improved cardiac function in heart failure and reduced in-stent neointima formation in the rat, but also improved systemic endothelial function.

Therefore, improved drug-eluting stents to reduce neointimal formation and improve cardiac function, such as stents eluting bioactive agents and Ang-(1-7) receptor agonists are needed.

### SUMMARY OF THE INVENTION

The present invention is directed providing medical devices, such as stents, with controlled-release drug-eluting polymer coatings capable of inhibiting restenosis and improving vascular endothelial cell function. Specifically, the vascular stents made in accordance with teachings of the present invention inhibit vascular smooth muscle cell proliferation, and therefore restenosis, by providing angiotensin-(1-7) (Ang-(1-7)) receptor agonists in combination with additional bioactive agents to the site of vascular injury.

In one embodiment of the present invention, a method is provided for inhibiting restenosis in a mammal comprising providing a vascular stent having a controlled-release coating thereon wherein the coating comprises an amphiphilic copolymer, an effective amount of at least one Ang-(1-7) receptor agonist and at least one additional bioactive agent; and inhibiting restenosis in the mammal. In another embodiment, the at least one Ang-(1-7) receptor agonist is Ang-(1-7) peptide in a concentration of between approximately 0.1% to 99% by weight of peptide-to-polymer.

In another embodiment, the vascular stent has a generally cylindrical shape comprising an outer surface, an inner surface, a first open end and a second open end and wherein at least one of the inner or outer surfaces are coated with the controlled-release coating. In another embodiment, the vascular stent further comprises a primer coat. In yet another embodiment, the amphiphilic copolymer comprises a PEG methacrylate-cyclohexyl methacrylate copolymer. In another embodiment, the vascular stent further includes a polymer topcoat comprising a PEG methacrylate-cyclohexyl methacrylate copolymer or poly(butyl methacrylate). In another embodiment, the vascular stent further comprises both a primer coat and a polymer topcoat.

In another embodiment of the present invention, the at least one bioactive agent is selected from the group consisting of FKBP 12 binding compounds such as zotarolimus, estrogens, chaperone inhibitors, protease inhibitors, protein-tyrosine kinase inhibitors, leptomycin B, peroxisome proliferator-activated receptor gamma ligands (PPARy), hypothemycin, bisphosphonates, epidermal growth factor inhibitors, antibodies, proteasome inhibitors, antibiotics, anti-inflammatories, anti-sense nucleotides and transforming nucleic acids. In yet another embodiment, the bioactive agent is rapamycin.

In another embodiment, the bioactive agent is present on the vascular stent in the same coating layer as the Ang-(1-7) receptor agonist. In another embodiment, the bioactive agent is present on the vascular stent in a different coating layer as the Ang-(1-7) receptor agonist.

In one embodiment of the present invention, a method is provided for improving endothelial cell function in a mammal comprising providing a vascular stent having a controlled-release coating thereon wherein the coating comprises an amphiphilic copolymer, an effective amount of at least one Ang-(1-7) receptor agonist and at least one additional bioactive agent; and improving vascular endothelial cell function in the mammal.

In one embodiment of the present invention, a medical device is provided comprising a stent having a generally cylindrical shape comprising an outer surface, an inner surface, a first open end and a second open end; a controlled-release coating comprising an amphiphilic copolymer, at least one Ang-(1-7) receptor agonist and at least one additional bioactive agent; wherein at least one of the inner or outer surfaces are adapted to deliver an effective amount of at least one Ang-(1-7) receptor agonist and at least one bioactive agent to a tissue of a mammal.

In another embodiment, the at least one Ang-(1-7) receptor agonist is a peptide having the amino acid sequence of SEQ ID NO. 1.

In another embodiment, the stent is a vascular stent.

In an embodiment of the present invention, at least one Ang-(1-7) receptor agonist is present on both the inner surface and the outer surface of the vascular stent. In another embodiment, the vascular stent further comprises a primer coat. In another embodiment, the amphiphilic copolymer comprises a PEG methacrylate-cyclohexyl methacrylate copolymer. In another embodiment, the medical device further includes a polymer topcoat comprising a PEG methacrylate-cyclohexyl methacrylate copolymer or poly(butyl methacrylate). In yet another embodiment, the vascular stent further comprises both a primer coat and a polymer topcoat. In another embodiment, the Ang-(1-7) peptide is in a concentration of between approximately 0.1% to 99% by weight of peptide-to-polymer.

### BRIEF DESCRIPTION OF THE DRAWINGS

FIG. 1 depicts the in vitro release profile of the Ang-(1-7)-eluting stent according to the teachings of the present invention.

FIG. 2 depicts the vasodilator function of isolated thoracic aortic rings in organ baths four weeks after stenting according to the teachings of the present invention. MeCh: metacholine. *; p<0.05 vs. bare metal, GLM repeated measures.

FIG. 3 depicts vascular responses in iliac arteries four weeks after stenting in rat abdominal aorta with bare metal stents according to the teachings of the present invention. Ang-(1-7) or saline was infused. PE: phenylephrine. MeCh: metacholine. *; p<0.05 vs. stent+Ang-(1-7) infusion and vs. sham, GLM repeated measures.

FIG. 4 depicts vascular responses in brachial arteries four weeks after abdominal stenting in rat abdominal aorta with bare metal stents according to the teachings of the present invention. Ang-(1-7) or saline was infused. PE: phenylephrine. MeCh: metacholine. *; p<0.05, vs. stent+saline infusion and vs. sham, GLM repeated measures.

FIG. 5 depicts the relative contribution of nitric oxide (NO), prostaglandins (PG) and endothelial-derived hyperpolarizing factor (EDHF) to the vasodilator effect of metacholine (10⁻⁴ mol/L) in isolated rat thoracic aortic rings, four weeks after sham operation or stenting according to the teachings of the present invention. Ang-(1-7) or saline was infused.

FIG. 6 depicts the effect of seven-day treatments (Ang-(1-7) and/or A779, both 10⁻⁷ mol/L) in cultured rat bone marrow mononuclear cells (MNC) and the subpopulation of endothelial progenitor cells (EPC) according to the teachings of the present invention. * p<0.05 vs. control, † p<0.05 vs. Ang-(1-7) alone.

FIG. 7 depicts the cumulative Ang-(1-7) release (FIG. 7A) and release rate (FIG. 7B) from Ang-(1-7)/rapamycin-coated stents according to the teachings of the present invention.

FIG. 8 depicts the cumulative rapamycin release (FIG. 8A) and release rate (FIG. 8B) from Ang-(1-7)/rapamycin-coated stents according to the teachings of the present invention.

### DEFINITION OF TERMS

Prior to setting forth the invention, it may be helpful to an understanding thereof to set forth definitions of certain terms that will be used hereinafter:

Animal: As used herein “animal” shall include mammals, fish, reptiles and birds. Mammals include, but are not limited to, primates, including humans, dogs, cats, goats, sheep, rabbits, pigs, horses and cows.

Bioactive agent: As used herein, “bioactive agents” shall include FKBP 12 binding compounds such as zotarolimus, rapamycin, estrogens, chaperone inhibitors, protease inhibitors, protein-tyrosine kinase inhibitors, leptomycin B, peroxisome proliferator-activated receptor gamma ligands (PPARγ), hypothemycin, bisphosphonates, epidermal growth factor inhibitors, antibodies, proteasome inhibitors, antibiotics, anti-inflammatories, anti-sense nucleotides and transforming nucleic acids. Bioactive agents can also include anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Biocompatible. As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero-order kinetics), that is the drug is released in even amounts over a predetermined time (with or without an initial burst phase) or may be a gradient release. A gradient release implies that the amount of drug released from the device surface changes over time.

Compatible: As used herein “compatible” refers to a composition possessing the optimum, or near optimum combination of physical, chemical, biological and drug release kinetic properties suitable for a controlled release coating made in accordance with the teachings of the present invention. Physical characteristics include durability and elasticity/ductility, chemical characteristics include solubility and/or miscibility and biological characteristics include biocompatibility. The drug release kinetics may be either near zero-order or a combination of first and zero-order kinetics.

Copolymer: As used here in a “copolymer” will be defined as ordinarily used in the art of polymer chemistry. A copolymer is a macromolecule produced by the simultaneous or step-wise polymerization of two or more dissimilar units such as monomers. Copolymer shall include bipolymer (two dissimilar units) terpolymer (three dissimilar units) etc.

Drug(s): As used herein “drug” shall include any bioactive compound having a therapeutic effect in an animal.

Treatment Site: As used herein “treatment site” shall mean a vascular occlusion, vascular plaque, an aneurysm site or other vascular-associated pathology.

### DETAILED DESCRIPTION OF THE INVENTION

The present invention is directed providing medical devices, such as stents, with controlled-release drug-eluting polymer coatings capable of inhibiting restenosis and improving vascular endothelial cell function. Specifically, the vascular stents made in accordance with teachings of the present invention inhibit vascular smooth muscle cell proliferation, and therefore restenosis, by providing bioactive agents to the site of vascular injury. Additionally, the coated medical devices of the present invention elute an agonist of the angiotensin-(1-7) (Ang-(1-7)) receptor which then improve vascular endothelial cell function.

In one embodiment of the present invention, an implanted medical device is provided with a polymer coating containing an agonist of the Ang-(1-7) receptor in combination with an additional bioactive agent. The present inventors have shown that the local administration of the agonist Ang-(1-7) from the surface of an implanted medical device significantly improves impaired vascular endothelial cell function. Coating of medical devices with Ang-(1-7) and biocompatible polymers is disclosed in co-pending U.S. patent application Ser. No. 11/256,582 filed Oct. 21, 2005 (now U.S. Pat. No. 7,176,261), which is incorporated herein by reference in its entirety.

The effect of Ang-(1-7) elution from a stent was assessed on neointima formation in the rat abdominal aorta and on endothelial function in thoracic aorta. Although neointima formation remained unaltered, upstream aortic endothelial function was improved by the Ang-(1-7)-eluting stent. Improvement of endothelial function after chronic Ang-(1-7) infusion was mainly due to increased prostaglandin (PG) and nitric oxide (NO) function.

In addition, Ang-(1-7) infusion improved endothelial function downstream of the stent (iliac artery) and in arteries distant from the stented area (brachial artery), and systemic endothelial function was associated with neointima formation. Ang-(1-7) treatment improved vasodilator function at all locations, but only endothelial function in upstream thoracic aorta had a significant negative correlation with in-stent neointima formation in the abdominal aorta.

To assess if stimulation of progenitor cells could be a target mechanism for systemic effects of Ang-(1-7), the effect on mononuclear cells (MNC) and endothelial progenitor cells (EPC) was studied. Exposure to Ang-(1-7) led to increased numbers of MNC in culture. The relative number of EPC was also increased by Ang-(1-7). The Ang-(1-7) receptor blocked by an Ang-(1-7) antagonist, A779, suggesting that the Mas receptor mediates these effects.

It has been shown in patient studies that the risk for restenosis in patients after stenting negatively correlates with endothelial function. The effect on thoracic aorta endothelial function, as seen after Ang-(1-7) minipump infusion, was reproduced by on-stent delivery of the Ang-(1-7) heptapeptide. Restoration of endothelial function and reduction of neointima formation are not the same process. The effect of Ang-(1-7) on neointima formation also depends on other qualities, e.g. an antiproliferative effect on vascular smooth muscle cells and an antithrombotic activity. These effects occur at different concentrations of Ang-(1-7), and therefore effects on endothelial function and reduction in neointima formation are not necessarily seen together.

Despite the absence of an effect of stent-delivered Ang-(1-7) on neointima formation, infusion of the peptide by minipump resulted in a negative correlation between upstream aortic dilator function and neointima formation. Therefore, upstream endothelial function may contribute to reduction of neointima formation.

Vascular injury by stenting has been postulated to cause endothelial dysfunction remote from the stent. There exists an inverse correlation between changes in C-reactive protein levels and flow-mediated brachial endothelial function 18 to 24 hours after percutaneous transluminal coronary intervention. Systemic endothelial function may be endangered because of release of inflammatory substances early after intervention. Hence, early protection could counteract this detrimental event.

Early therapy with Ang-(1-7) seems to provide a very potent protection. Very early after onset of infusion of Ang-(1-7), hypotensive responses to acetylcholine are improved in an NO-dependent manner. This rapid NO effect on endothelium might protect against functional decline or even apoptosis. Alternatively, improved repair of systemic endothelium, either by local endothelial proliferation or supply from the pool of progenitor cells, can mediate protection. Local repair be improved by the rapid NO-mediated effect.

The present inventors have also observed that Ang-(1-7) stimulates bone marrow-derived EPC. Bolus injections of Ang-(1-7) increase bone marrow restoration after irradiation or chemotherapy as measured by recovery of hematopoiesis in rodents and humans. The present inventors have demonstrated that Ang-(1-7) has a broad spectrum of activities that also includes effects on progenitor cells such as EPC. This finding holds with the observation that Angiotensin II (Ang II) may impair EPCs and that countering the activity of Ang II can be beneficial.

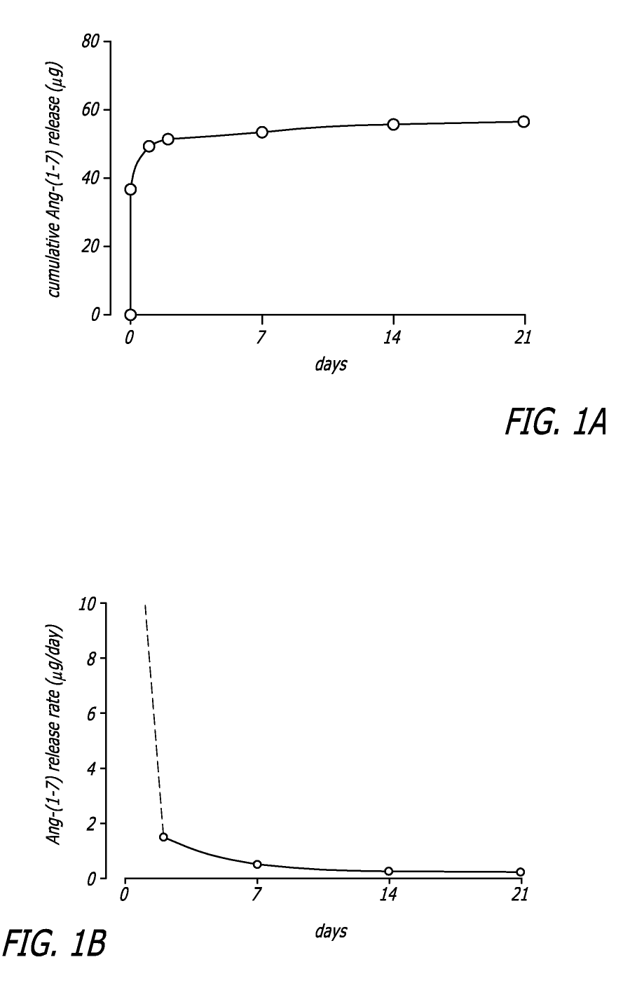

Ang-(1-7) may mediate these endothelium effects. Ang-(1-7)-coated stents demonstrate an elution profile of second order kinetics (FIG. 1) in which Ang-(1-7) is initially released in a high amount, equivalent to a bolus injection of 40 μg, followed by a longer-lasting constant infusion of 1 μg/day, equivalent to 0.7 ng/min, for 21 days. It has been shown previously that a dose of 0.7 μmol/min of Ang-(1-7), an equivalent of 0.6 ng/min, improves endothelial function. The intravascular bolus injection of 40 μg is well over the bolus dose of 100 μg/kg given subcutaneously in previous studies to stimulate bone marrow recovery. Hence, this dose is likely to have a bioactive effect, even on bone marrow cells.

When looking at the mechanism of changes in aortic endothelial function with respect to signal factors, stenting mainly leads to a decrease in NO signaling, leaving endothelium-derived hyperpolarizing factor (EDHF) function intact. Chronic Ang-(1-7) infusion leads to appearance of PG-mediated relaxation function. Additionally, NO release is increased such that the relative contribution to total endothelial dilator function remains equal as compared to that after stenting. EDHF function does not improve. The effect of chronic Ang-(1-7) infusion on endothelial function is different than when chronic angiotensin converting enzyme (ACE) inhibition or angiotensin 11 type 1 receptor (AT₁) blockade is applied. Chronic ACE or AT₁ inhibition leads to improved EDHF function or NO rather than PG.

In summary, the present invention demonstrates that an Ang-(1-7)-eluting stent can be used as a drug delivery devices to improve endothelial function. This improvement can take place either through improvement of PG and EDHF function, or through blunting of adrenergic contractile function.

Furthermore, in a one embodiment of the present invention, the Ang-(1-7) eluting stents can be administered with additional bioactive agents. Suitable bioactive agents include, but are not limited to, FKBP 12 binding compounds such as zotarolimus, rapamycin, estrogens, chaperone inhibitors, protease inhibitors, protein-tyrosine kinase inhibitors, leptomycin B, peroxisome proliferator-activated receptor gamma ligands (PPARγ), hypothemycin, bisphosphonates, epidermal growth factor inhibitors, antibodies, proteasome inhibitors, antibiotics, anti-inflammatories, anti-sense nucleotides and transforming nucleic acids. The bioactive agents can be delivered to the treatment site by incorporation into the biocompatible polymer coating on the vascular stent or can be administered systemically by administration methods known to persons of ordinary skill in the art.

The polymeric coatings of the present invention can be applied to stent surfaces, either primed or bare, in any manner known to those of ordinary skill in the art. Application methods compatible with the present invention include, but are not limited to, spraying, dipping, brushing, vacuum-deposition, and others. Moreover, the polymeric coatings may be used with a cap coat. A cap coat as used herein refers to the outermost coating layer applied over at least one other coating. At least one drug-containing polymer coating is applied over the primer coat. Then, a polymer cap coat is applied over the drug-containing polymeric coating. The cap coat may optionally serve as a diffusion barrier to control the release of the drug(s). The cap coat may be merely a biocompatible polymer applied to the surface of the stent to protect the stent and have no effect on the drug release rates.

### Example 1

### Ang-(1-7)-Eluting Stent

Ang-(1-7)-eluting stents were made from polyurethane-primed Medtronic Driver® stents (2.5×8 mm) spray-coated using a 0.5 wt. % PEG-methacrylate polymer solution in methyl alcohol containing 20 wt. % Ang-(1-7). Next, a topcoat was applied by spray-coating using a poly(butylmethacrylate) (PBMA) solution in chloroform. Ang-(1-7) elution from ethylene oxide (EtO)-sterilized stents after 1 hour, and 1, 2, 7, 14, and 21 days was determined in vitro in a buffer at 37° C. with fluorimetric quantification using fluorescamine.

The amount of the PEG-methacrylate coating applied as a base coat on the stents was 461±43 μg, containing 93±9 μg Ang-(1-7). The weight of the PBMA topcoat was 369±50 μg. Smooth coatings were obtained showing good durability during crimping, EtO-sterilization, tracking and expanding the stents. The in vitro release profile was determined, and the stents showed an initial burst in the first 24 hours followed by a continuous slow release for up to 21 days (FIG. 1).

### Example 2

### Effects of Ang-(1-7) on Stented Endothelium

Specific pathogen-free, male Wistar rats (Harlan, Horst, Netherlands) were fed standard rat chow and water ad libitum. For Ang-(1-7) administration by minipump, 28 rats were used. The animals were anaesthetised with O₂, N₂O and isoflurane (Abbot B.V., Hoofddorp, Netherlands). In 20 animals, bare metal stents (Medtronic Driver® 2.5×8 mm stents) were placed in the abdominal aorta. Upon stent placement, osmotic minipumps (Model 2004, Alzet, Charles River, Maastricht, The Netherlands) were implanted subcutaneously for drug delivery via a catheter in the jugular vein. The pumps infused either Ang-(1-7) (Bachem, Well am Rhein, Germany) (24 μg/kg/h) (n=10) or saline (0.25 μl/h) (n=10). Furthermore, a control group underwent sham surgery (n=8: surgery identical to stent groups, but without balloon insertion and stenting), and received an osmotic minipump releasing saline. Treatment continued for four weeks. Five rats were excluded because of technical failure (aortic leakage or minipump failure) resulting in n=7 for Ang-(1-7) infusion, n=10 for saline infusion, and n-6 for sham-operated rats.

To test the effect of Ang-(1-7)-eluting stents (see Example 1 for details), 36 rats underwent placement of three types of stents into the abdominal aorta, bare metal stents, stents coated with polymer alone, or stents coated with polymer containing approximately 90 μg Ang-(1-7). Six rats were excluded because of technical failure during surgery, and three stents could not be processed due to incomplete polymerisation of embedding plastic (see below) leaving n=9 for bare metal stent, n=10 for stent with polymer only and n=8 for stent with polymer+Ang-(1-7).

Twenty-eight days after stent implantation, the animals were anaesthetised with O₂, N₂O and isoflurane, and systemically heparinized with 500 IE intravenously (Leo Pharma B.V., Breda, The Netherlands). The stented abdominal aortas were harvested, fixed, embedded in methylmethacrylate, sectioned and stained for histological analysis as described previously. The thoracic aorta, left iliac and left brachial arteries were removed and placed in a Krebs solution (pH 7.5) containing (mM): NaCl (120.4), KCl, (5.9), CaCl₂ (2.5), MgCl₂ (1.2), NaH₂PO₄ (1.2), glucose (11.5), NaHCO₃, (25.0).

The stented aortas were evaluated in organ bath studies with isolated vascular rings. The thoracic aorta was carefully prepared in Krebs solution, and peri-aortic tissue was removed from the thoracic aorta and rings of approximately 2 mm length were cut. The rings were connected to an isotonic displacement transducer at a preload of 14 mN in an organ bath containing Krebs solution at 37° C. and continuously bubbled with 95% O₂ and 5% CO₂. After stabilization for 60 minutes, during which Krebs solution was regularly refreshed, rings were checked for viability by stimulation with phenylephrine (PE; 1 mM). The rings were washed and restabilized. Sets of rings were precontracted with PE (1 mM) to induce maximal contraction (Eₘₐₓ). The endothelium-dependent vasodilation was assessed by a cumulative dose of metacholine (MeCh; 10 nM to 0.1 mM). To assess the contributions of NO, vasodilator prostaglandins PG and EDHF to the total endothelial function, a cumulative dose of MeCh was administered in presence of NO-synthase inhibitor N^{G}-monomethyl-L-arginine (L-NMMA) (100 mM), with or without the prostaglandin synthesis inhibitor indomethacin (10 mM). The NO-dependent vasodilation to metacholine was calculated, by subtraction of the vasodilation in the presence of L-NMMA from the total vasodilation. The PG-dependent vasodilation in response to MeCh was calculated by subtraction of the vasodilation in the presence of L-NMMA and indomethacin from the vasodilation in the presence of L-NMMA. The EDHF-dependent vasodilation was determined by the vasodilation to metacholine in the presence of L-NMMA and indomethacin. Subsequently, the rings were dilated maximally with the endothelium-independent vasodilator sodium nitrite (10 mM).

Iliac and brachial arteries were prepared under a dissection microscope in Krebs solution that was kept at 4° C. Rings were cut and had an average length of 2.5±0.3 mm for iliac arteries and 2.3±0.4 mm for brachial arteries. The rings were mounted in a small vessel myograph (EMKA Technologies, Paris, France) onto two tungsten wires (ø 25 μm, Advent Research Materials, Halesworth, England). The rings were left to equilibrate at zero tension at 37° C. in bubbled Krebs solution for 45 minutes during which three washing steps were performed. After this, vessels were brought to tension in 20 μm steps until reaching a diameter corresponding to 100 mmHg transmural pressure (L₁₀₀, calculated by exponential curve fitting with Excel software). The diameter was then set on 90% of L₁₀₀, and the rings were equilibrated for 45 minutes, and all subsequent measurements were performed isometrically at this diameter. Cumulative doses of PE (10⁻⁹ to 10⁻⁶ mol/L) were administered, and the response to 10⁻⁶ mol/L PE was considered as Eₘₐₓ. Then, PE was washed out and rings were equilibrated for 45 minutes, while the concentration of PE to obtain 70% of the Eₘₐₓ (EC₇₀) as calculated by means of sigmoidal curve fitting. After equilibration, vessels were precontracted with PE until EC₇₀ and cumulative doses of MeCh (10⁻⁹ to 10⁻⁶ mol/L) were added. All responses were recorded in grams tension. The responses to MeCh were expressed as the percent decrease in PE EC₇₀ responses.

To determine if Ang-(1-7) infusion by minipump or elution from the stent reduced neointima formation, neointima area was measured in animals which received Ang-(1-7) either by stent or minipump (Table 1). Ang-(1-7) eluted from stents did not have an effect on neointimal as compared to stents coated with polymer alone, whilst the polymer itself increased neointima formation as compared to bare metal stents (Table 1). Infusion of Ang-(1-7) using a minipump resulted in a reduction in neointimal formation.

TABLE 1

| | | | | Regression statistics | | | | | |
|---|---|---|---|---|---|---|---|---|---|
| | | | | NIA vs. Eₘₐₓ | | | | | |
| | | | | aorta | | a. iliacis | | a. brachialis | |
| | Treatment | Neointima area | | (n = 16) | | (n = 16) | | (n = 12) | |
| Delivery method | protocol | NIA (μm²) | N | r² | p | r² | p | r² | p |
| Minipump | saline | 0.70 ± 0.07 | 10 | 0.28 | <.05 | 0.22 | NS | 0.05 | NS |
| Infusion¹ | Ang-(1-7) | 0.51 ± 0.05* | 7 | | | | | | |
| Stent-based | bare metal | 0.68 ± 0.04 | 9 | | | | | | |
| | polymer | 0.97 ± 0.06^{†} | 10 | | | | | | |
| | Polymer + | 0.98 ± 0.08^{†} | 8 | | | | | | |
| | Ang-(1-7) | | | | | | | | |

*p < 0.05 vs. saline;
^{†}p < 0.01 vs. bare metal.

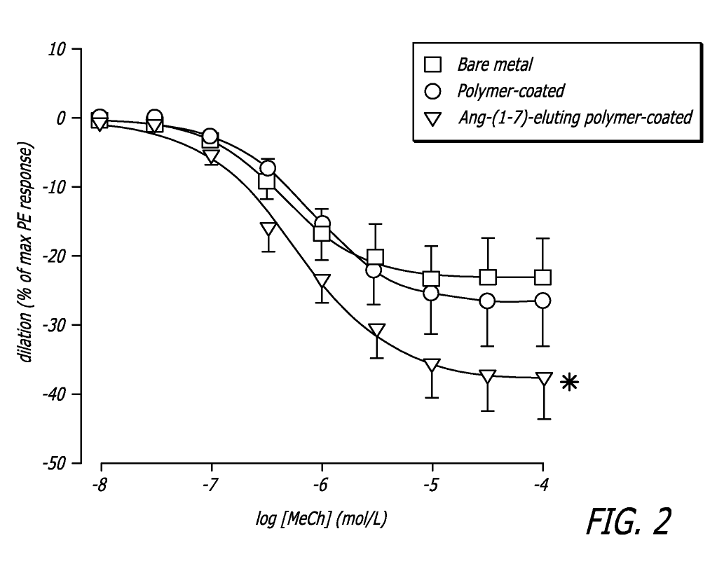

To determine if Ang-(1-7) elution had an effect on endothelial function, the vasodilator responses of isolated thoracic aorta rings to metacholine was tested in an organ bath. In rats implanted with the Ang-(1-7)-eluting stent, endothelial function was significantly improved as compared to bare stents (FIG. 2). The same trend was seen as compared to stents coated with polymer alone, but did not reach statistical significance. The stent coated with polymer alone did not have an effect on endothelial function.

The literature provides evidence that stent placement worsens systemic endothelial function, and possibly also small arteries downstream of the stent. The Ang-(1-7)-coated stent of the present invention was effective in improving endothelial function. To determine if Ang-(1-7) would also be effective in smaller arteries, either downstream of the stent or in compartments of the body remote to the stented segment, the endothelial function was determined in the iliac artery (downstream) and the brachial artery (remote) in rats stented with bare metal stents in which Ang-(1-7) was administered by osmotic minipump.

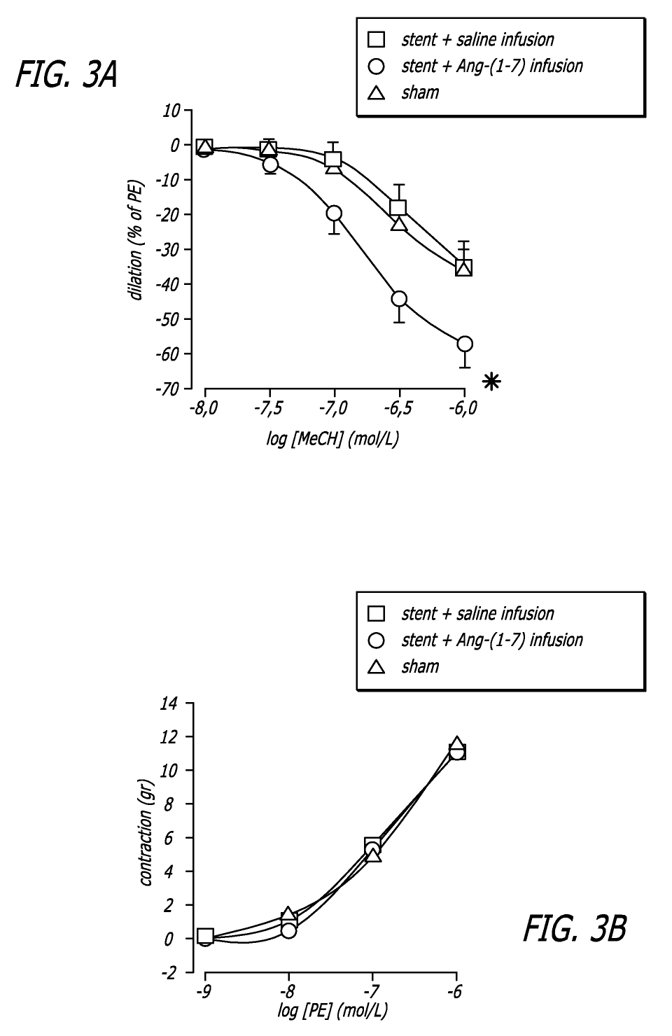

Endothelial function in the iliac artery did not change after stenting as compared to sham-operated rats (FIG. 3). However, chronic infusion of Ang-(1-7) by minipump led to increased vasodilation upon stimulation with metacholine. Vasoconstrictions remained equal after stenting or Ang-(1-7) treatment (FIG. 3).

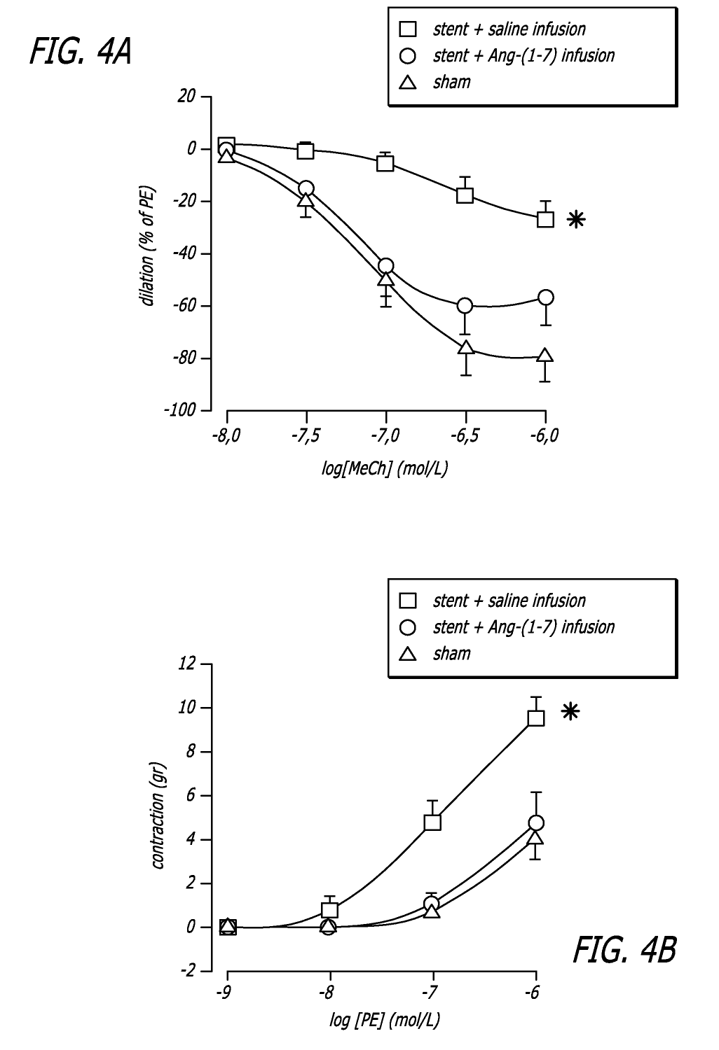

In the remotely located brachial artery, vasodilator function was severely decreased after stent placement, but Ang-(1-7) infusion improved vasodilator function (FIG. 4). Unlike aorta and iliac artery, vasoconstrictor responses in the brachial artery to phenylephrine were increased after stenting. Ang-(1-7) decreased vasoconstrictor responses to the level of sham-operated rats.

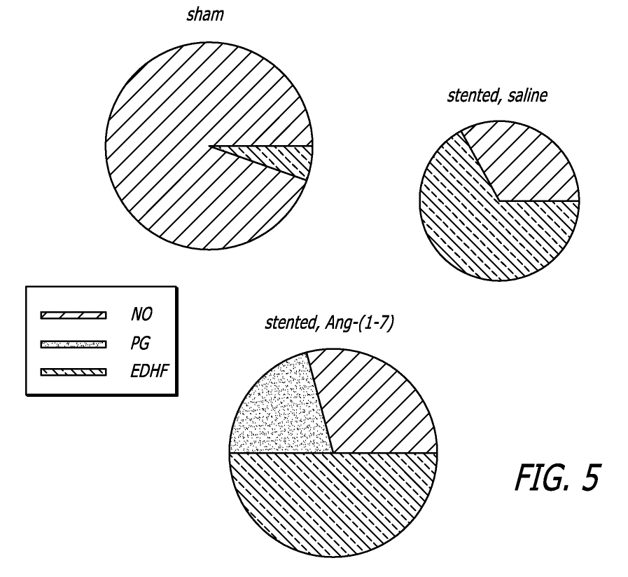

Endothelium-dependent vasodilatation depends primarily on the three signaling factors NO, PG and EDHF. The relative contribution of these factors was assessed in the thoracic aorta of sham-operated and stented rats treated with saline or Ang-(1-7) by minipump infusion. The dose-response curves for the total response on metacholine showed a 40% decrease in Eₘₐₓ in the stented group, and a full recovery after Ang-(1-7) infusion (represented by the size of the pies in FIG. 5). In sham-operated rats dilations depended almost exclusively on NO (FIG. 5), leaving a small contribution to EDHF and no role at all for PG. Stenting led to a major decrease in NO release, being partly rescued by increased EDHF. Ang-(1-7) treatment did not alter NO release, but stimulated both PG and EDHF.

The above data suggest that firstly, improvement of endothelial function is associated with neointima formation and, secondly, that the endothelial function was differentially altered when comparing downstream arterial function to upstream and remote arterial function. Linear regression analyses were performed to assess a putative correlation between maximal vasodilator response and neointima formation as far as both parameters were available for each individual animal.

Maximal response to metacholine thoracic aorta significantly correlated with neointimal area in the stented abdominal aorta in a negative trend, explaining 28% of the variation in neointima area (Table 1). In contrast to aortic dilator function, maximal metacholine responses in brachial and artery did not significantly correlate with neointima area.

### Example 3

### The Effect of Ang-(1-7) on Cultured Rat EPC

In recent literature, evidence has emerged supporting a role of bone marrow-derived stem cells, and more specifically, endothelial progenitor cells, in the process of neointima formation and endothelial maintenance. Given the evident systemic action of Ang-(1-7), its effect on bone marrow-derived mononuclear cells (MNC) and endothelial progenitor cells (EPC) was tested.

Five rats were sacrificed by exsanguination under O₂, N₂O and isoflurane anaesthesia. Bone marrow was isolated from the left and right femurs by flushing the bone marrow cavity with sterile phosphate-buffered saline at room temperature (PBS, Gibco, Invitrogen, Breda, The Netherlands). From each individual rat, MNC were obtained by density gradient centrifugation at 2000 rpm (MSE Mistral 3000i, UK) for 20 minutes at room temperature according to manufacturer's instruction (Cedarlane Laboratories ltd., Hornby, Canada). Cells from individual rats were then resuspended in endothelial cell basal medium-2 (EGM; Cambrex Bioproducts, Clonetics, New Jersey, USA) containing supplemented 2% fetal bovine serum and EGM-2 SingleQuots (Clonetics). At day 1, 5×10⁵ cells/well were seeded in 1% gelatin (225 bloom, Sigma-Aldrich, Zwijndrecht, The Netherlands) precoated 96-well culture plates (Costar, Corning, N.Y., USA) at 37° C. in a humidified incubator in the presence of 5% CO₂. At day 3 of culture, medium with non-attached cells was removed, and replaced by fresh culture medium containing Ang-(1-7) at 10⁻⁷ mol/L with or without antagonists (treatment medium). Next, cells were grown for 7 days, changing treatment medium at day 5 and day 7. At day 9, treatment medium was removed and culture medium supplemented with 10 μg/mL acetylated low-density lipoprotein (Dil-Ac-LDL) was added to the cells for 4 h. Thereafter, cells were fixed with 4% paraformaldehyde in phosphate-buffered saline (pH 7.5) at 4° C. for 10 minutes. Then, cells were stained with Bandeiraea simplicifolia 1 lectin (BSI) (Sigma, the Netherlands) and 4′,6-diamidino-2-phenylindole nuclear stain (DAPI, Molecular Probes, Leiden, the Netherlands).

Detection of staining was performed with the use of fluorescence microscopy (Leica, Wetzlar, Germany) at a magnification of 200×. From each well, pictures from high power fields at 5 random locations that formed an imaginary “x” were taken, taking in consideration a comparable distribution of the detection loci between all wells. Cells were quantified with the use of ImagePro software (Media Cybernetics, Silver Spring, USA). In the occasion that the presence of cell clusters hindered proper digital quantification, cell numbers were assessed by hand. Triple-stained cells were defined as EPC while all DAPI-stained mononuclear cells were defined as MNC.

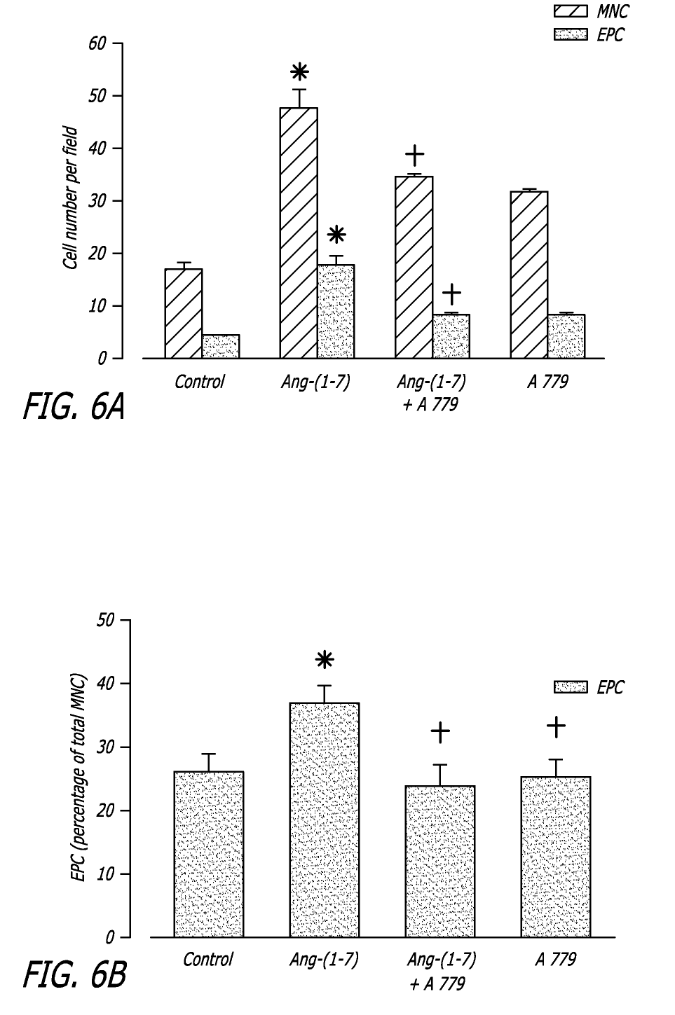

To this purpose MNC were isolated from rat bone marrow and cultured for 2 days to allow attachment. Thereafter, cells were treated with Ang-(1-7) (10⁻⁷ mol/L), and/or its antagonist A779 (10⁻⁷ mol/L). Ang-(1-7) increased the number of MNC and EPC after 7 days of culture (FIG. 6A). The effect of Ang-(1-7) was blocked by A779. A779 itself also increased the number of MNC and EPC, but Ang-(1-7) had less effect than A779 alone. To determine if Ang-(1-7) preferentially stimulated EPC, the number of EPC was expressed as a percentage of the total population of MNC (FIG. 6B). Ang-(1-7) increased the relative number of EPC, and this effect was fully blocked by A779. A779 alone had no effect on the relative number of EPC.

### Example 4

### Design of Angiotensin-(1-7)/Rapamycin Eluting Stent

An Ang-(1-7)/rapamycin eluting stent comprised a first coating layer of an amphiphilic copolymer containing 10% by weight of Ang-(1-7) and a second coating layer comprising 20% by weight rapamycin. The second coating provided an additional barrier capable of providing sustained release of Ang-(1-7).

A hexylmethacrylate/hydroxyethylmethacrlylate (HMA/HEMA) copolymer (see co-pending U.S. patent application Ser. No. 10/970,171, which is incorporated by reference herein for all its discloses regarding polymers) was dissolved in methanol (MeOH) at room temperature overnight while shaking to a concentration of approximately 0.5% (wt/vol). Ang-(1-7) was added to the dissolved copolymer so that a concentration of 10% by weight of Ang-(1-7) relative to the HMA/HEMA copolymer was achieved. In a similar manner a PBMA/rapamycin solution in chloroform was prepared with a concentration of 20% by weight of rapamycin relative to the PBMA polymer.

The (HMA/HEMA) copolymer/Ang-(1-7) solution was sprayed on 18 mm polyurethane-primed Medtronic Driver® stents using standard spraying equipment in which the copolymer solution was vaporized ultrasonically. Coating weights were approximately 600 μg per stent, containing 60 μg Ang-(1-7). Next, the PBMA second coating containing rapamycin was applied. Coating weights were around 300 μg per stent, containing 60 μg rapamycin.

### Example 5

### Release of Angiotensin-(1-7)/Rapamycin from Coated 18 mm Stents in Vitro

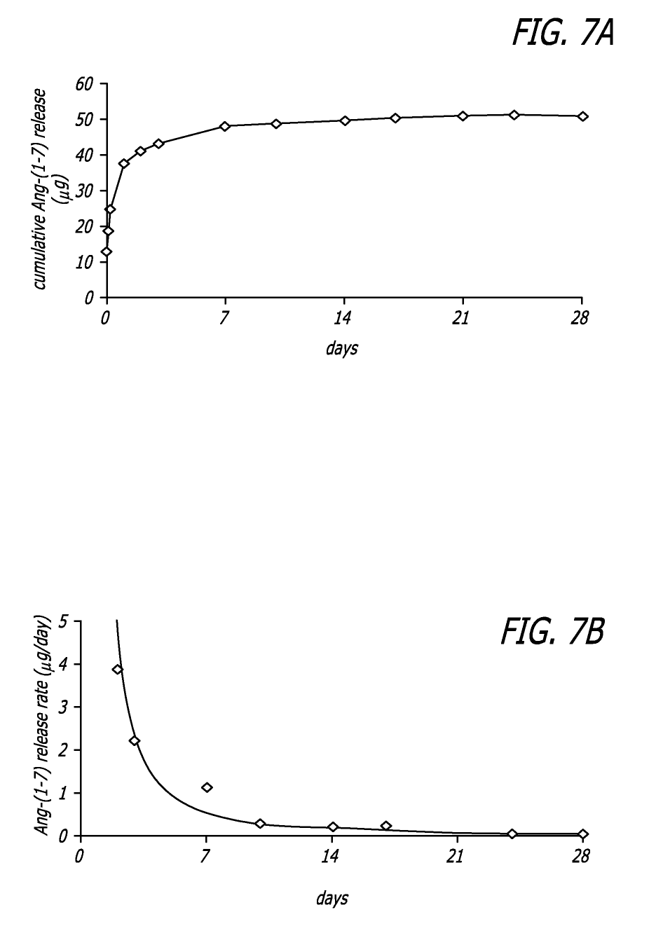

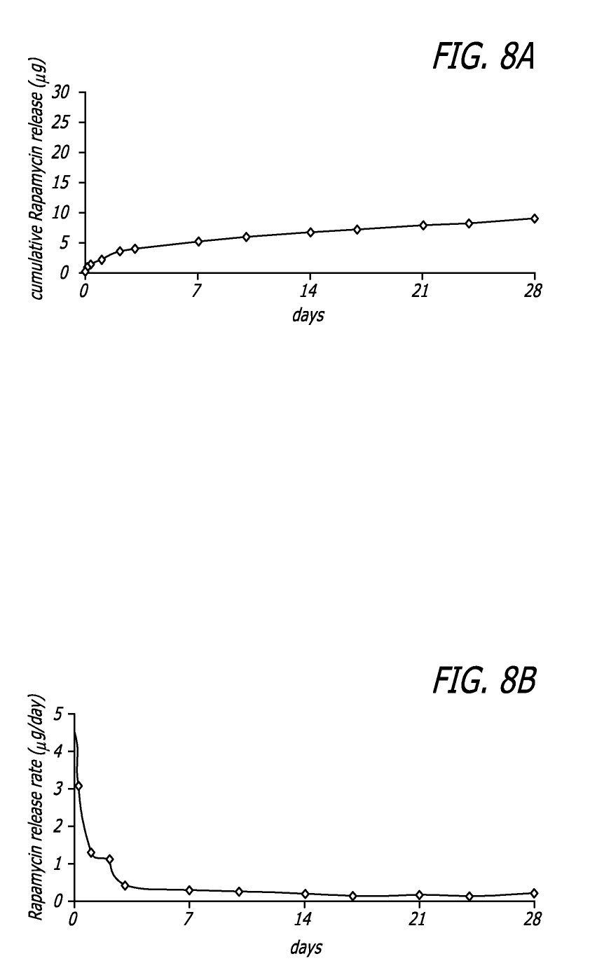

For an in vitro study of Ang-(1-7) release from coated stents, a series of 18 mm stents described in Example 4 were used. The results of the in vitro release study are presented in FIGS. 7 and 8. The Ang-(1-7) release tests were performed in triplicate in phosphate-buffered saline (PBS) (pH 7.4) at 37° C. for periods up to 28 days. The stents were incubated in 750 μL of PBS containing sodium azide and at specific times the stents were removed from the PBS and the releasing media was analyzed for Ang-(1-7) using standard fluorescence techniques. The rapamycin release tests were performed in triplicate in Tris-buffer (10 mM, pH 7.4) comprising SDS (0.4% by weight) at 37° C. for periods up to 28 days. The stents were incubated in 1500 μL of Tris-buffer containing sodium azide and at specific times the stents were removed from the Tris-buffer and the releasing media was analyzed for rapamycin using standard HPLC with UV-detection techniques.

The stents demonstrated a 40% burst of Ang-(1-7) and a sustained release of Ang-(1-7) for 28 days. The release rate at day 14 was approximately 0.3 μg/day (FIG. 7). Additionally the stents showed a sustained release of rapamycin as shown in FIG. 8.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for inhibiting restenosis in a mammal comprising:
providing a vascular stent having a controlled-release coating thereon, said coating comprising
an amphiphilic copolymer,
an effective amount of an Ang-(1-7) peptide, and
at least one additional bioactive agent selected from the group consisting of FKBP 12 binding compounds such as zotarolimus, estrogens, chaperone inhibitors, protease inhibitors, protein-tyrosine kinase inhibitors, leptomycin B, peroxisome proliferator-activated receptor gamma ligands (PPARy), hypothemycin, bisphosphonates, epidermal growth factor inhibitors, antibodies, proteasome inhibitors, antibiotics, anti-inflammatories, anti-sense nucleotides and transforming nucleic acids; and
inhibiting restenosis in said mammal.

2. The method according to claim 1 wherein said peptide comprises SEQ ID NO: 1.

3. (canceled)

4. The method according to claim 1 wherein said vascular stent further comprises a primer coat.

5. The method according to claim 1 wherein said amphiphilic copolymer comprises a PEG methacrylate-cyclohexyl methacrylate copolymer.

6. The method according to claim 1 wherein said vascular stent further includes a polymer topcoat comprising a PEG methacrylate-cyclohexyl methacrylate copolymer or poly(butyl methacrylate).

7. The method according to claim 1 wherein said vascular stent further comprises both a primer coat and a polymer topcoat.

8. The method according to claim 1 wherein said bioactive agent is rapamycin.

9. (canceled)

10. (canceled)

11. A method for improving endothelial cell function in a mammal comprising:
providing a vascular stent having a controlled-release coating thereon, said coating comprising
an amphiphilic copolymer,
an effective amount of an Ang-(1-7) peptide, and
at least one additional bioactive agent selected from the group consisting of FKBP 12 binding compounds such as zotarolimus, estrogens, chaperone inhibitors, protease inhibitors, protein-tyrosine kinase inhibitors, leptomycin B, peroxisome proliferator-activated receptor gamma ligands (PPARy), hypothemycin, bisphosphonates, epidermal growth factor inhibitors, antibodies, proteasome inhibitors, antibiotics, anti-inflammatories, anti-sense nucleotides and transforming nucleic acids; and
improving at least one vascular endothelial cell function selected from the group consisting of improved vasodilation, improved vasoconstriction and increased production of endothelial progenitor cells in said mammal.

12. (canceled)

13. (canceled)

14. (canceled)

15. The method according to claim 11 wherein said vascular stent further comprises a primer coat.

16. The method according to claim 11 wherein said amphiphilic copolymer comprises a PEG methacrylate-cyclohexyl methacrylate copolymer.

17. The method according to claim 11 wherein said vascular stent further includes a polymer topcoat comprising a PEG methacrylate-cyclohexyl methacrylate copolymer or poly(butyl methacrylate).

18. The method according to claim 11 wherein said vascular stent further comprises both a primer coat and a polymer topcoat.

19. The method according to claim 11 wherein said bioactive agent is rapamycin.

20. A medical device comprising:
a stent having a generally cylindrical shape comprising an outer surface, an inner surface, a first open end and a second open end;
a controlled-release coating comprising an amphiphilic copolymer, an Ang-(1-7) peptide and at least one additional bioactive agent selected from the group consisting of FKBP 12 binding compounds such as zotarolimus, estrogens, chaperone inhibitors, protease inhibitors, protein-tyrosine kinase inhibitors, leptomycin B, peroxisome proliferator-activated receptor gamma ligands (PPARy), hypothemycin, bisphosphonates, epidermal growth factor inhibitors, antibodies, proteasome inhibitors, antibiotics, anti-inflammatories, anti-sense nucleotides and transforming nucleic acids;
wherein at least one of said inner or outer surfaces are adapted to deliver an effective amount of said Ang-(1-7) peptide and said at least one bioactive agent to a tissue of a mammal.

21. (canceled)

22. (canceled)

23. The medical device of claim 20 wherein said Ang-(1-7) peptide is present on both said inner surface and said outer surface of said vascular stent.

24. The medical device of claim 20 wherein said vascular stent further comprises a primer coat.

25. The medical device of claim 20 wherein said amphiphilic copolymer comprises a PEG methacrylate-cyclohexyl methacrylate copolymer.

26. The medical device of claim 20 wherein said medical device further includes a polymer topcoat comprising a PEG methacrylate-cyclohexyl methacrylate copolymer or poly(butyl methacrylate).

27. The medical device of claim 20 wherein said vascular stent further comprises both a primer coat and a polymer topcoat.
